(19) United States
(12) Patent Application Publication
(10) Pub. No.: US 2009/0131902 A1
(43) Pub. Date: May 21, 2009

(54) Disposable Undergarment with a Front Opening Panel

(75) Inventor: Merav Giloh, Hod HaSharon (IL)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: TAMICARE LTD., Manchester (GB)

(21) Appl. No.: 12/268,212

(22) Filed: Nov. 10, 2008

Related U.S. Application Data
(60) Provisional application No. 61/002,724, filed on Nov. 9, 2007.

(51) Int. Cl. A61F 13/15

(52) U.S. Cl. 604/385.22; 604/386; 604/396

(57) ABSTRACT

A disposable undergarment with a front opening panel is described. The undergarment includes a waist band having an inner surface, an outer surface, a front side, a back side, a top portion, and a bottom portion, and a panel extending from the bottom portion of the back side of the waist band, the panel having a first end, a front portion, and a back portion. The first end of the panel is secured to the waist band by a suitable securing mechanism. In another embodiment, the waist band includes a first end and a second end, wherein the first end of the waist band is attached to the second end of the waist band by another suitable securing mechanism.

### RELATED APPLICATIONS

This application claims priority to Provisional Application Ser. No. 61/002,724, which was filed on Nov. 9, 2007.

### FIELD

The application relates to a disposable undergarment with a front opening panel.

### BACKGROUND

Disposable undergarments with open panels are well known in the art, such as baby diapers and incontinence briefs. The undergarments are produced as a flattened product with the rear and front panels detached. In order to wear the product, the rear panel is placed on the backside of the wearer, the front panel is brought through the crotch area to the front side of the wearer, and the front and rear panels are secured by overlapping front and rear panel ends and attaching them together with different types of adhesives, on the waist sides or mid front of the wearer.

The main disadvantage of these products is that when the potential wearer is standing, it is complicated to put on the product and to wear the product so it fits properly. In cases where the person dresses themselves, it becomes even almost impossible. The solution for this disadvantage given by some manufacturers of such products is for the wearer to lean with their backside against a stable surface, such as a wall, and temporarily secure the rear panel to the back side of their body while the front panel is brought forward to its place. Since most of the self-dressing wearers for these products are elderly users, this dressing method is far from comfortable.

In order to overcome the above disadvantage, users may close the product prior to wearing it. Kimberly Clark, for example, sells a baby diaper which is ready to use as a pre-closed product where the tapes can be re-opened, in order to ease baby diaper changing. The same solution applies to adults and babies. However, when the user wears panties, trousers, or pantyhose, that solution is impossible unless the user takes off the garment (panties, trousers or pantyhose), puts on the pre-closed undergarment/diaper, and then puts the garment on again. This process is complicated and creates discomfort especially when the changing of the undergarment is taking place in a public lavatory.

The need to wear an “open product” also arises with women wearing disposable underwear during menstruation while wearing trousers or pantyhose.

### SUMMARY

The present application offers a new aesthetically designed and more comfortable method for wearing disposable undergarments such as panties, knickers, briefs, and diapers. In one embodiment, the application discloses an undergarment comprising a waist band having an inner surface, an outer surface, a front side, a back side, a top portion, and a bottom portion, and a panel extending from the bottom portion of the back side of the waist band, the panel having a first end, a front portion, and a back portion. The first end of the panel is secured to the waist band by a second securing mechanism.

In another embodiment, the application discloses an undergarment comprising a waist band having a first end and a second end, and a panel extending from the bottom portion of the back side of the waist band, the panel having a first end, a front portion, and a back portion. In this embodiment, the first end of the waist band is attached to the second end of the waist band by a first securing mechanism and the bottom portion of the waist band is secured to the first end of the panel by a second securing mechanism.

A method for putting on an undergarment is also described. The method includes providing a waist band including a front side, a back side, a top portion and a bottom portion, providing a panel extending from the bottom portion of the back side of the waist band, the panel having a first end, putting on the waist band, pulling the first end of the panel toward the front side of the waist band, and securing the panel to the waist band with a securing mechanism.

### BRIEF DESCRIPTION OF THE DRAWINGS

Exemplary embodiments of the invention are described herein with reference to the drawings, in which:

FIG. 1 shows one embodiment of an undergarment in an open position;

FIG. 2 shows the undergarment of FIG. 1 in a closed position;

FIG. 3 shows another embodiment of an undergarment in a fully open position;

FIG. 4 shows the undergarment of FIG. 3 with the waist band in a closed position;

FIG. 5 shows the undergarment of FIG. 3 with the closed waist band and in process of being fully closed;

FIG. 6 shows the undergarment of FIG. 3 in the closed position;

FIG. 7 shows another embodiment of the designed product at a fully open position;

FIG. 8 shows the product of FIG. 5 with the waist band in a closed position;

FIG. 9 shows the product of FIG. 5 with the closed waist band and in process of being fully closed; and

FIG. 10 shows the product of FIG. 5 in the closed position.

### DETAILED DESCRIPTION

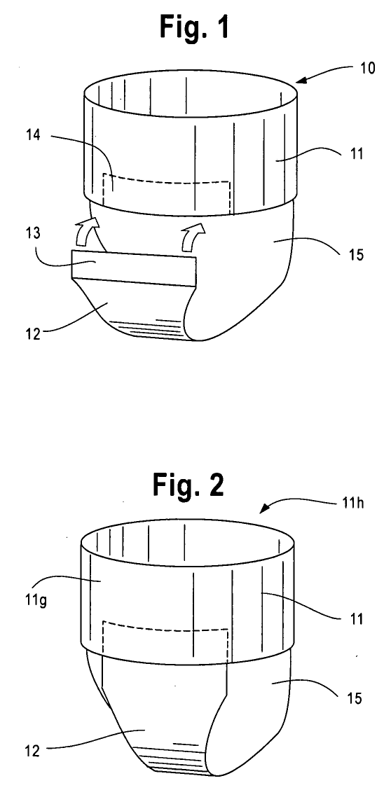

Referring to FIG. 1, an undergarment 10 is provided. The undergarment 10 is made of one piece of material created by an upper part of the undergarment back panel 15, a front panel 12, and a waist band 11. Thus, the front of the undergarment includes a waist opening and a temporary opening in the bottom. This allows the wearer to first pull up the closed waist band over their pulled down undergarment (trousers, knickers, panties etc.) onto their body, so the waist band holds the garment on the wearer's body and no additional support for holding the undergarment on the body is needed.

The undergarment 10 includes a waist band 11. The waist band 11 can be constructed in different ways. In one embodiment, shown in FIGS. 1-2, the waist band 11 is a single circular piece already closed and ready to be pulled up. In this embodiment, the waist band 11 may include reusable adhesive tabs (not shown), so that the wearer is able to adjust the undergarment on their body after putting it on to suit their specific size.

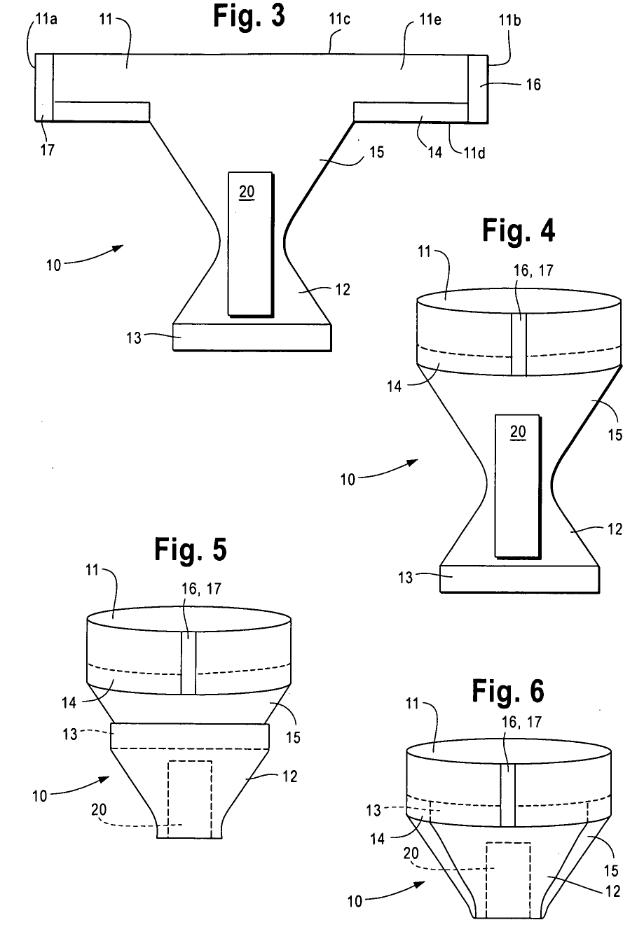

In another embodiment, as shown in FIG. 3, the waist band 11 is open with two ends and the wearer attaches the ends together to create the closed waist band. In this embodiment, the first end 11a and second end 11b are secured to each other by a first securing mechanism 16, 17. The first securing mechanism 16, 17 may comprise double-sided tape. Alternatively, the first securing mechanism may be any suitable securing means, such as Velcro, adhesive, or snaps.

In both embodiments, the waist band 11 further includes a top portion 11c and a bottom portion 11d. The waist band 11 also includes a front side 11g which rests against the wearer's navel and a back side 11h which rests against the wearer's back. The bottom portion 11d of the waist band may include a one end of a second securing mechanism 14. The front panel 12 may include the other end of the second securing mechanism 13. The second securing mechanism 13, 14 may be located on an inner surface 11e or an outer surface 11f of the waist band 11, as shown in FIGS. 3 and 4. The second securing mechanism 13, 14 may comprise double-sided tape. Alternatively, the second securing mechanism may be any suitable securing means, such as Velcro, adhesive, or snaps.

The undergarment 10 further includes a panel section having a front panel 12 and a back panel 15 extending from the bottom portion 11d of the waist band 11. The panel 12, 15 may have an hourglass shape. The front panel 12 may be folded to attach to the bottom portion 11d of the waist band 11 by the second securing mechanism 13, 14. The second securing mechanism 13, 14 may either be located on either side of the front panel 12, or on the inner 11e or outer 1 if surfaces of the waist band 11.

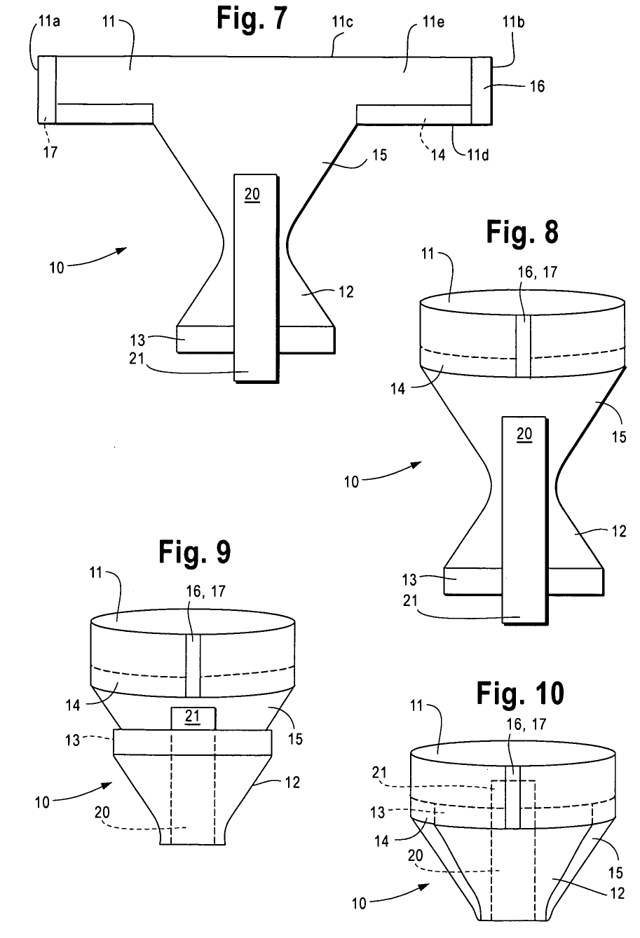

The undergarment may further include a pad 20, or any other suitable absorptive device, preferably located on the front panel section 12 of the undergarment 10. In one embodiment the pad 20, which is unified with the undergarment, is located entirely within the panel 12 of the undergarment 10, as shown in FIGS. 3-6. In another embodiment, shown in FIGS. 7-10, a pad 21 extends partially outside of the front panel 12. The adhesive side of the pad 21 creates an additional securing means to adhere the front panel 12 to the waist band 11.

In operation, the undergarment 10 shown in FIGS. 1 and 2 can be worn by pulling the closed waist band 11 over the wearer's knees and also over their bottom item of clothing while sitting or standing. Then, the panel 12, 15 of the undergarment, which is temporarily hanging from the back side of the waist band 11, can be brought forward via the crotch area and secured to the waist band 11 on the front side of the undergarment 10 by any suitable securing mechanism 13, 14. The leg openings are created when the panel 12, 15 of the undergarment is secured to the waist band 11. This method allows easier mobility wearing the undergarment while standing up, laying down, or sitting.

In the embodiments depicted in FIGS. 3-10, the first end 11a and second end 11b of the waist band 11 are brought together and secured by securing mechanism 16, 17. Next, the front panel 12 is brought to the front between the legs and attached to the waist band, so that the product is ready to be pulled up to its place.

The undergarment 10 may be made out of an elastic or stretchable material. The undergarment can be made as one layer with the open end front panel. Alternatively, the undergarment can be made out of more then one sheet, and can be combined out of several same or different materials or sheets. The undergarment can also be made out of any suitable materials such as cloth, elastomer, polymer or the like. Additional suitable materials that may be used with this application include natural rubber (e.g. latex), synthetic rubber, a fiber based material, or any materials mentioned in U.S. Pat. Nos. 6,987,210, 7,354,424, and U.S. Patent Publication No. 2008/0150191, which are all hereby incorporated by reference in their entirety.

If natural rubber, synthetic rubber or any other elastic or stretchable polymeric material is used to make undergarment 10, at least part or all of the surface of the material is preferably flocked with any suitable loose fibers such as cotton fibers or viscose fibers to create a smooth and cozy feeling for the wearer. Suitable flocking methods are described, for example, in U.S. Pat. Nos. 6,987,210 and 7,354,424; U.S. Patent Publication 2006/0113714; and U.S. application No. 2008/0150191, all which are hereby incorporated by reference in their entirety.

The undergarment 10 may be manufactured by a number of different methods, including the methods described in detail in U.S. Pat. Nos. 6,987,210, 7,354,424, and 6,960,740; U.S. Patent Publication Nos. 2006/0113714 and 2008/0150191; and U.S. patent Ser. No. 12/053,040, all which are hereby incorporated by reference in their entirety.

The present design allows the discharge of the undergarment by easily tearing the undergarment in the attaching regions, such as by detaching securing mechanisms 13, 14 between the panel and the waist band, and by detaching securing mechanisms 16, 17 on the waist band 11.

The present figures represent the basic concept of the innovative design which is not limited to the specific shape and style, or according to gender styles.

While certain features and embodiments of the present application have been described in detail herein, it is to be understood that the application encompasses all modifications and enhancements.

What is claimed:

1. A disposable undergarment comprising:
a waist band having an inner surface, an outer surface, a front side, a back side, a top portion, and a bottom portion; and
a panel extending from the bottom portion of the back side of the waist band, the panel having a first end, a front portion, and a back portion;
wherein the first end of the panel is secured to the waist band by a securing mechanism.

2. The disposable undergarment of claim 1 wherein the waist band comprises a first end and a second end; and
wherein the first end of the waist band is attached to the second end of the waist band by a second securing mechanism.

3. The disposable undergarment of claims 1 or 2 wherein the securing mechanism is located on the inner surface of the bottom portion of the waist band.

4. The disposable undergarment of claims 1 or 2 wherein the securing mechanism is located on the outer surface of the bottom portion of the waist band.

5. The disposable undergarment of claims 1 or 2 further comprising an absorptive device secured to an inside portion of the panel.

6. The disposable undergarment of claim 5 wherein the absorptive device is located entirely within the panel.

7. The disposable undergarment of claim 5 wherein the absorptive device is located partially within the panel.

8. The disposable undergarment of claim 5 wherein the absorptive device is an absorptive pad.

9. The disposable undergarment of claims 1 or 2 wherein the undergarment is made of an elastic material.

10. The disposable undergarment of claims 1 or 2 wherein the panel has an hourglass shape.

11. A method for putting on a disposable undergarment comprising:
providing a waist band including a front side, a back side, a top portion and a bottom portion;
providing a panel extending from the bottom portion of the back side of the waist band, the panel having a first end;
putting on the waist band;
pulling the first end of the panel toward the waist band; and
securing the panel to the waist band with a securing mechanism.
